(19) United States
(12) Patent Application Publication
(10) Pub. No.: US 2021/0290237 A1
(43) Pub. Date: Sep. 23, 2021

(54) COLONIC PROSTHESIS FOR INTRALUMINAL FECAL DIVERSION

(71) Applicant: Covidien LP, Mansfield, MA (US)

(72) Inventors: Arvind Rajagopalan Mohan, Dracut, MA (US); Travis Rowe, Santa Rosa, CA (US); Gene Yang, Buffalo, NY (US)

(21) Appl. No.: 17/191,064

(22) Filed: Mar. 3, 2021

Related U.S. Application Data
(60) Provisional application No. 62/993,228, filed on Mar. 23, 2020.

(51) Int. Cl. A61B 17/11

(52) U.S. Cl. CPC A61B 17/1114; A61B 2017/1132

(57) ABSTRACT

A colonic prosthesis is configured for intraluminal fecal diversion at the site of an anastomosis to reduce anastomotic leaks. The colonic prosthesis includes circular waveform structures secured to each other by a graft material that is formed of impermeable material. The colonic prosthesis has a sinusoidal profile to enable transition between compressed configuration and expanded configuration to facilitate insertion into the colon.

### CROSS-REFERENCE TO RELATED APPLICATION

This application claims the benefit of and priority to U.S. Provisional Patent Application No. 62/993,228, filed Mar. 23, 2020, the entire disclosure of which is incorporated by reference herein.

### FIELD

The disclosure relates generally to surgical instruments for use in gastrointestinal surgery, and more particularly, to colonic prostheses for intraluminal fecal diversion.

### BACKGROUND OF RELATED ART

A stent is an elongated device used to support a luminal wall. Various types of stents include many designs that encompass a filament or number of filaments, such as a wire or wires, wound into a particular configuration to help maintain a lumen open. In another type of stent, a graft material is provided on a frame of the stent to help reduce a leak through the material.

### SUMMARY

In accordance with the disclosure, a colonic prosthesis includes a plurality of circular waveform structures arranged along a length of the colonic prosthesis and a graft material extending between a proximal-most circular waveform structure to a distal-most circular waveform structure. The plurality of circular waveform structures is transitionable between a compressed configuration and an expanded configuration. Each circular waveform structure of the plurality of circular waveform structures includes struts and crowns. Each crown interconnects adjacent struts. The graft material defines a lumen through the plurality of circular waveform structures.

In an aspect, the colonic prosthesis may include first and second end portions and a middle portion interconnecting the first and second end portions. The circular waveform structures in the first and second end portions may be disposed closer to each other than the circular waveform structures disposed in the middle portion.

In another aspect, each circular waveform structure of the plurality of circular waveform structures may have a sinusoidal profile.

In yet another aspect, at least one circular waveform structure of the plurality of circular waveform structures may have struts of different lengths.

In still yet another aspect, the plurality of circular waveform structures may be formed of a wire.

In still yet another aspect, the circular waveform structures may be uniformly spaced apart along a length of the colonic prosthesis.

In an aspect, the plurality of circular waveform structures may be configured to exert a radial force in a range between about 9.33 N and about 45.6 N.

In another aspect, the plurality of circular waveform structures may be transitionable between a first diameter and a second diameter larger than the first diameter.

In yet another aspect, the plurality of circular waveform structures may have a diameter in a range between about 18 mm and about 33 mm.

In still yet another aspect, the graft material may be formed of impermeable material.

In still yet another aspect, the graft material may conform to a shape of the proximal-most circular waveform structure or the distal-most circular waveform structure.

In an aspect, the graft material may be infused with a medicament.

In accordance with another aspect of the disclosure, a colonic prosthesis includes a tubular graft material and a plurality of circular waveform structures arranged along a length of the tubular graft material. The plurality of circular waveform structures is transitionable between a compressed configuration and an expanded configuration. Each circular waveform structure of the plurality of circular waveform structures includes struts and crowns. Each crown interconnects adjacent struts.

In an aspect, the colonic prosthesis may further include a suture configured to fasten an end portion of the tubular graft material to retain at least one circular waveform structure of the plurality of circular waveform structures in the compressed configuration.

In another aspect, the colonic prosthesis may further include a wire secured to an end portion of the tubular graft material. The wire may be configured to be crimped to retain at least one circular waveform structure of the plurality of circular waveform structures in the compressed configuration.

In an aspect, the tubular graft material may have a length of about 5 cm.

In another aspect, the circular waveform structures may be uniformly arranged along the length of the tubular graft material.

In yet another aspect, the tubular graft material may be formed of an impermeable material.

In an aspect, the plurality of circular waveform structures may be formed of shape memory material.

In another aspect of the disclosure, a method of surgery includes introducing a colonic prosthesis into a lumen of a colon in a compressed configuration; positioning the colonic prosthesis at a site of an anastomosis such that the colonic prosthesis extends across the site of the anastomosis; and radially expanding the colonic prosthesis to an enlarged circumference, thereby placing the outer surface of the colonic prosthesis in contact with an inner wall of the colon across the site of the anastomosis, thereby anchoring the colonic prosthesis within the colon. The colonic prothesis includes a plurality of circular waveform structures arranged along a length of the colonic prosthesis, and a graft material extending between a proximal-most circular waveform structure to a distal-most circular waveform structure, thereby defining a lumen through the plurality of circular waveform structures. The plurality of circular waveform structures has a diameter in a range between about 18 mm and about 33 mm. Each circular waveform structure of the plurality of circular waveform structures includes struts and crowns. Each crown interconnects adjacent struts.

In an aspect, introducing the colonic prosthesis may include placing the colonic prothesis through a delivery device.

In another aspect, positioning the colonic prosthesis may include positioning the colonic prosthesis at the site of the anastomosis after formation of the anastomosis.

In yet another aspect, radially expanding the colonic prosthesis may include exerting a radial force in a range between about 9.33 N and about 45.6 N by the plurality of circular waveform structures.

### BRIEF DESCRIPTION OF THE DRAWINGS

Various aspects of the disclosure are illustrated herein with reference to the accompanying drawings, wherein:

FIG. 1 is a perspective view of a colonic prosthesis in accordance with the disclosure;

FIG. 2 is a perspective view of the colonic prosthesis of FIG. 1, illustrating use with a suture;

FIG. 3 is a perspective view of a colonic prosthesis in accordance with another aspect of the disclosure;

FIG. 4 is a perspective view of a colonic prosthesis in accordance with yet another aspect of the disclosure;

FIG. 5 is a perspective view of the colonic prosthesis of FIG. 4 and a delivery device for use with the colonic prosthesis;

FIG. 6 is a perspective view of the colonic prosthesis of FIG. 3, illustrating use thereof with a suture;

FIG. 7 is a perspective view of the colonic prosthesis of FIG. 4, illustrating use with a crimping wire; and

FIG. 8 is a flow chart illustrating a method of fecal diversion.

### DETAILED DESCRIPTION

A colonic prosthesis is described in detail with reference to the drawings, wherein like reference numerals designate corresponding elements in each of the several views. As used herein, the term “distal” refers to that portion of the instrument, or component thereof which is farther from the user while the term “proximal” refers to that portion of the instrument or component thereof which is closer to the user.

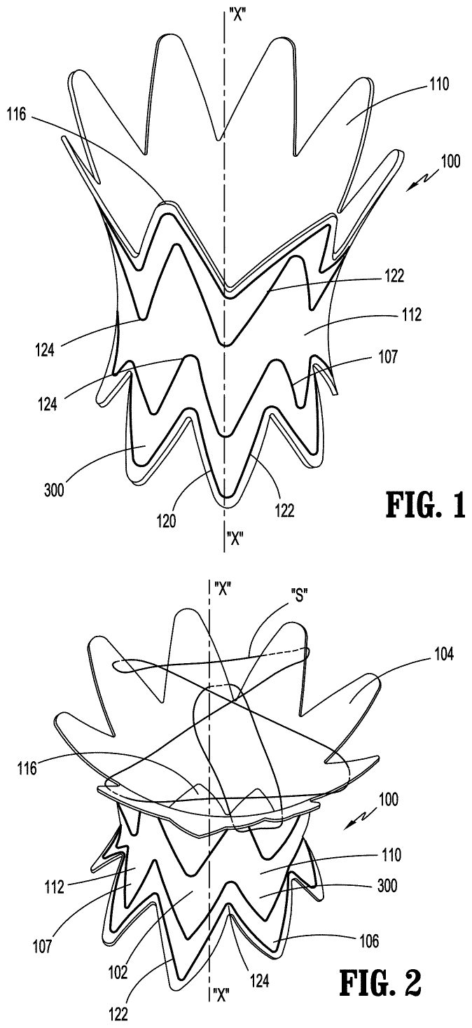

FIG. 1 illustrates a colonic prosthesis 100 for use in colorectal surgical procedures. As used herein with reference to the disclosure, colorectal surgical procedures encompass surgeries of the colon, rectum and anus, and refer to procedures including end-to-end anastomoses of the colon to remove diseased sections thereof. In particular, the colonic prosthesis 100 may be suitable for, e.g., intraluminal fecal diversion, at the site of an anastomosis to reduce anastomotic leaks. The colonic prosthesis 100 may reduce the risk of anastomotic leaks and inhibit tissue growth at the site of the anastomosis. The colonic prosthesis 100 may enhance healing by providing suitable radial pressure to the colon or by providing a drug-infused body.

The colonic prosthesis 100 includes a hollow tubular member 110 having a wall 112 defining a lumen 116 along its longitudinal axis “X-X”. The lumen 116 may have a diameter in a range between about 18 mm and about 33 mm. In particular, the diameter may be about 23 mm. In another aspect, the diameter may be about 25 mm. In another aspect, the diameter may be about 28 mm. In another aspect, the diameter may be about 30 mm. The colonic prosthesis 100 may have a length of about 5 cm. The colonic prosthesis 100 may provide a radial force in a range between about 9.33 N and about 45.6 N. In an aspect, the radial force may be about 21.4 N. In another aspect, the radial force may be about 27.2 N. In yet another aspect, the radial force may be about 29 N.

FIGS. 1 and 2 illustrate the colonic prosthesis 100 having a first end portion 104, a second end portion 106, and a middle or central portion 102 disposed between the first and second end portions 104, 106. In particular, the first and second end portions 104, 106 may be flared out to enhance, e.g., securement within the intestinal lumen. The wall 112 of the colonic prosthesis 100 has a layer 300 formed of, e.g., a graft material, to inhibit fecal matter from being trapped in the cells/openings of the colonic prosthesis 100. It is contemplated that the layer 300 may only extend a length of the colonic prosthesis 100.

The colonic prosthesis 100 is formed from wires 120 wrapped around in a tubular waveform shape around the longitudinal axis “X-X” to form the colonic prosthesis 100. In particular, a plurality of circular waveform structures 107 is arranged along the longitudinal axis “X-X.” The plurality of circular waveform structures 107 may be secured to each other by the layer 300. The colonic prosthesis 100 is expandable, i.e., it may be introduced into the intestinal lumen with a reduced circumference (e.g., a compressed configuration), and thereafter radially expands to an enlarged circumference (e.g., an expanded configuration), which places the outer surface of the colonic prosthesis 100 in contact with the inner wall of the intestine at the site of placement, thereby anchoring the colonic prosthesis 100 in place at the desired location within the intestine. The colonic prosthesis 100 may be infused with one or more medicaments to promote healing of tissue and inhibit infection of tissue.

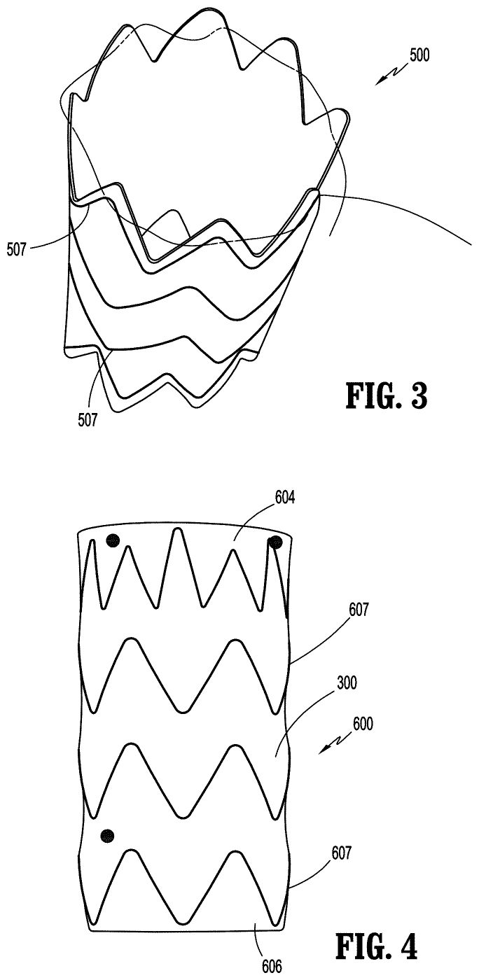

The wire 120 may be made of any material, such as titanium, tantalum, gold, copper or copper alloys, nickel-titanium alloys, combinations of these materials, or any other biologically compatible material used in stents as known to those skilled in the art. The wire 120 is formed into a tubular waveform including a series of straight segments or struts 122 interconnected by bends or crowns 124. The tubular waveform is, e.g., substantially a sinusoid. However, as would be understood by those skilled in the art, other waveforms may be utilized. Further, the amplitude need not be a constant amplitude. For example, the length of struts 112 may alternate between a short strut and a long strut (see, e.g., FIG. 4). Further, the lengths of the struts 112 may be varied. In particular, the crowns 124 along the length of the colonic prosthesis 100 may be in registration with each other. The circular waveform structures 107 may be disposed closer to each other in the first and second end portions 104, 106 and may be spaced apart from each other in the middle portion 102. However, it is contemplated that, as shown in FIG. 3, a colonic prosthesis 500 may include circular waveform structures 507 that are uniformly spaced apart along a length of the colonic prosthesis 500.

The colonic prosthesis 100 may be shaped or formed from a material that can be provided with a mechanical memory to return the structure from a compressed or constricted delivery configuration to an expanded deployed configuration. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or colonic prosthesis structure by thermal treatment to achieve a spring temper in stainless steel, for example, or to set a shape memory in a susceptible metal alloy, such as nitinol. Various polymers that can be made to have shape memory characteristics may also be suitable for use. For example, polymers such as polynorborene, trans-polyisoprene, styrene-butadiene, and polyurethane may be utilized. In addition, poly L-D lactic copolymer, oligo caprylactone copolymer and poly cyclo-octine may be used separately or in conjunction with other shape memory polymers.

In addition, the colonic prosthesis 100 may be braided from filaments formed of suitable self-expanding, shape memory or superelastic material. The braid formed of such shape memory materials is heat-treated or “heat-set” at a high temperature in order to reduce internal stresses in the filaments and/or increase or impart a self-expanding capability of the colonic prosthesis. Filaments making up the tubular body of the colonic prosthesis 100 that has been heat set are in their least-stressed or a reduced-stressed state when the colonic prosthesis 100 is in the configuration it was in during heat setting. Such a least-stressed or reduced-stressed state can include an expanded or fully expanded state, thereby making the colonic prosthesis self-expanding.

As shown in FIGS. 1 and 2, the layer 300 in the first and second end portions 104, 106 conform to the shape and curvature of the circular waveform structure 107. However, it is contemplated that the layer 300 of the colonic prosthesis 600 (FIG. 4) may extend beyond proximal-most and distal-most circular waveform structures 507 in first and second end portions 504, 506 to define a tubular profile. The layer 300 may have extremely low permeability to facilitate intraluminal fecal diversion. In particular, the layer 300 may be impermeable to, among other things, alimentary tract contents such as, for example, bile, water, and/or fecal matter. This inhibits the openings/cells of the colonic prosthesis 100 from becoming clogged with fecal matter, as well as reduces leakage at the site of the anastomosis, thereby imparting impermeability to the colonic prosthesis 100. The layer 300 may be made from any elastomeric polymer capable of expanding with the colonic prosthesis 100. Non-limiting examples of suitable elastomeric polymers include natural or synthetic rubbers, polyurethane, polyisoprene, polybutadiene, chloroprene, polyisobutylene, as well as combinations and copolymers thereof.

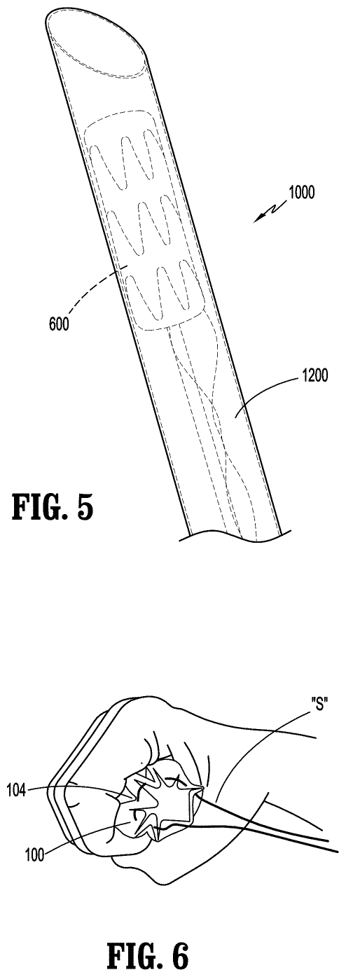

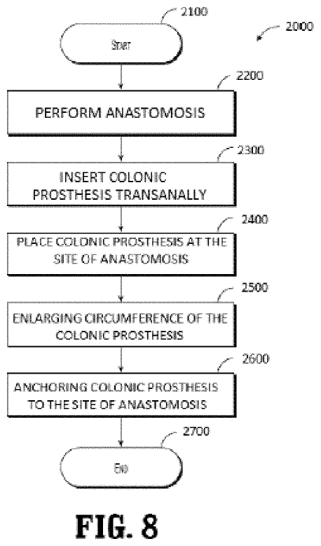

In use, FIG. 8 shows a flow chart for performing fecal diversion 2000 with the colonic prosthesis 100, 500, 600 of the disclosure. The procedure starts at step 2100. At step 2200, the colonic prosthesis 100, 500, 600 is introduced into a colon in a compressed configuration by using a delivery device 1000 (FIG. 5) that has a tubular structure 1200. In particular, the tubular structure 1200 of the delivery device 1000 is configured to receive a colonic prosthesis 100, 500, 600 in the compressed configuration. At step 2300, an anastomosis is performed on a target tissue. Once the anastomosis has been performed at step 2300, the colonic prosthesis 100, 500, 600 may be introduced to the site of the anastomosis by the delivery device 1000 in step 2400. At this time, the colonic prosthesis 100, 500, 600 extends across the site of the anastomosis. It is contemplated that the colonic prosthesis 100, 500, 600 may be placed at the site of the anastomosis while performing the anastomosis. At step 2500, the colonic prosthesis 100, 500, 600 is radially expanded to an enlarged circumference, thereby placing the outer surface of the colonic prosthesis 100, 500, 600 in contact with an inner wall of the colon across the site of the anastomosis, thereby anchoring the colonic prosthesis within the colon at step 2600. The process ends at step 2700 when the colonic prosthesis 100, 500, 600 is securely anchored to the colon.

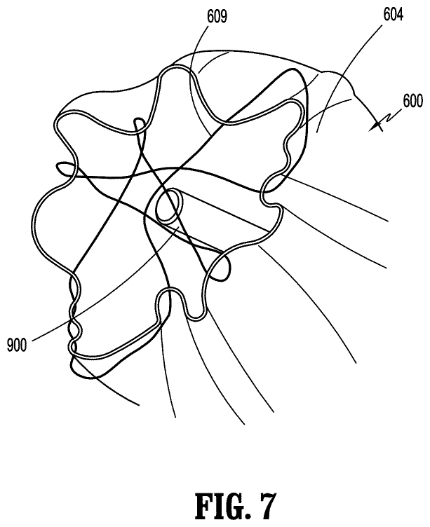

In order to facilitate insertion of the colonic prosthesis 100, 500, 600 into the delivery device 1000, the clinician may utilize a suture “S” (FIG. 6) to fasten the respective first and second end portions 104, 106 of the colonic prosthesis 100 to reduce the diameters thereof. While FIG. 6 utilizes the suture “S” with the colonic prosthesis 100, it is contemplated that the suture “S” may be used with the colonic prostheses 500, 600. Alternatively, the colonic prosthesis 600 as shown in FIG. 7 may include a crimping wire 609 secured around the first and second end portions 604, 606 (only the first end portion 604 shown). The circumferentially arranged crimping wire 609 may be crimped together using a crimping tool 900 to transition the colonic prosthesis 600 into a radially compressed configuration to facilitate insertion through the delivery device 1000. While the crimping wire 609 has been shown for use with colonic prosthesis 600, it is contemplated that the crimping wire 609 may be utilized with the colonic prostheses 100, 500. After delivery to the treatment site, the suture “S” may be unfastened or the crimping wire 609 may be removed such that the colonic prosthesis 100, 500, 600 is permitted to expand to its radially expanded configuration to provide appropriate radial force. The radial force exerted by the colonic prosthesis 100, 500, 600 is sufficient to secure the colonic prosthesis 100, 500, 600 within the colon. It is contemplated that the colonic prostheses 100, 500, 600 may be deployed within the colon at the time of performing the anastomosis or deployed after the anastomosis.

The description provided herein may refer to the deployment of the colonic prosthesis 100, 500, 600 in particular to the colon and rectum, but it is also understood that aspects of the disclosure may be employed within other parts of the gastrointestinal tract, such as the small intestine, biliary tract, esophagus, and/or stomach.

Persons skilled in the art will understand that the devices and methods specifically described herein and illustrated in the accompanying drawings are non-limiting. It is envisioned that the elements and features may be combined with the elements and features of another without departing from the scope of the disclosure. As well, one skilled in the art will appreciate further features and advantages of the disclosure.

1. A colonic prosthesis comprising:
a plurality of circular waveform structures arranged along a length of the colonic prosthesis, the plurality of circular waveform structures transitionable between a compressed configuration and an expanded configuration, each circular waveform structure of the plurality of circular waveform structures including struts and crowns, each crown interconnecting adjacent struts; and
a graft material extending between a proximal-most circular waveform structure to a distal-most circular waveform structure, thereby defining a lumen through the plurality of circular waveform structures.

2. The colonic prosthesis according to claim 1, wherein the colonic prosthesis includes first and second end portions and a middle portion interconnecting the first and second end portions, the circular waveform structures in the first and second end portions are disposed closer to each other than the circular waveform structures disposed in the middle portion.

3. The colonic prosthesis according to claim 1, wherein each circular waveform structure of the plurality of circular waveform structures has a sinusoidal profile.

4. The colonic prosthesis according to claim 2, wherein at least one circular waveform structure of the plurality of circular waveform structures has struts of different lengths.

5. The colonic prosthesis according to claim 1, wherein the plurality of circular waveform structures is formed of a wire.

6. The colonic prosthesis according to claim 1, wherein the plurality of circular waveform structures is uniformly spaced apart along a length of the colonic prosthesis.

7. The colonic prosthesis according to claim 1, wherein the plurality of circular waveform structures is configured to exert a radial force in a range between about 9.33 N and about 45.6 N.

8. The colonic prosthesis according to claim 1, wherein the plurality of circular waveform structures is transitionable between a first diameter and a second diameter larger than the first diameter.

9. The colonic prosthesis according to claim 1, wherein the plurality of circular waveform structures has a diameter in a range between about 18 mm and about 33 mm.

10. The colonic prosthesis according to claim 1, wherein the graft material is formed of impermeable material.

11. The colonic prosthesis according to claim 1, wherein the graft material conforms to a shape of the proximal-most circular waveform structure or the distal-most circular waveform structure.

12. The colonic prosthesis according to claim 1, wherein the graft material is infused with a medicament.

13. A colonic prosthesis comprising:
a tubular graft material; and
a plurality of circular waveform structures arranged along a length of the tubular graft material, the plurality of circular waveform structures transitionable between a compressed configuration and an expanded configuration, each circular waveform structure of the plurality of circular waveform structures including struts and crowns, each crown interconnecting adjacent struts.

14. The colonic prosthesis according to claim 13, further comprising a suture configured to fasten an end portion of the tubular graft material to retain at least one circular waveform structure of the plurality of circular waveform structures in the compressed configuration.

15. The colonic prosthesis according to claim 13, further comprising a wire secured to an end portion of the tubular graft material, the wire configured to be crimped to retain at least one circular waveform structure of the plurality of circular waveform structures in the compressed configuration.

16. The colonic prosthesis according to claim 13, wherein the tubular graft material has a length of about 5 cm.

17. A method of surgery comprising:
introducing a colonic prosthesis into a lumen of a colon in a compressed configuration, the colonic prothesis including:
a plurality of circular waveform structures arranged along a length of the colonic prosthesis, the plurality of circular waveform structures having a diameter in a range between about 18 mm and about 33 mm, each circular waveform structure of the plurality of circular waveform structures including struts and crowns, each crown interconnecting adjacent struts; and
a graft material extending between a proximal-most circular waveform structure to a distal-most circular waveform structure, thereby defining a lumen through the plurality of circular waveform structures;
positioning the colonic prosthesis at a site of an anastomosis such that the colonic prosthesis extends across the site of the anastomosis; and
radially expanding the colonic prosthesis to an enlarged circumference, thereby placing the outer surface of the colonic prosthesis in contact with an inner wall of the colon across the site of the anastomosis, thereby anchoring the colonic prosthesis within the colon.

18. The method according to claim 17, wherein introducing the colonic prosthesis includes placing the colonic prothesis through a delivery device.

19. The method according to claim 17, wherein positioning the colonic prosthesis includes positioning the colonic prosthesis at the site of the anastomosis after formation of the anastomosis.

20. The method according to claim 17, wherein radially expanding the colonic prosthesis includes exerting a radial force in a range between about 9.33 N and about 45.6 N by the plurality of circular waveform structures.
